(19) United States
(12) Patent Application Publication
(10) Pub. No.: US 2004/0054310 A1
(43) Pub. Date: Mar. 18, 2004

(54) Supportive knee brace

(76) Inventor: Abol Behrooz Dadfarmay, Greenwood, IN (US)

Correspondence Address: Abol B. Dadfarmay, 467 Van Hoy Dr., Greenwood, IN, 46142, US

(21) Appl. No.: 10/244,173

(22) Filed: Sep. 16, 2002

(51) Int. Cl. A61F 5/00

(52) U.S. Cl. 602/26

(57) ABSTRACT

The SUPPORTIVE KNEE BRACE is an eighteenth inch brace made with soft material called neoprene. The brace is a front closure style and when wrapped around the knee it exposes the patella. The edge of the brace has a piece of velcro attached to each edge longitudinally in an equal length. When they are overlapping on the mid fine of the knee, it securely holds the brace in a tight position. The two straps above and below the knee joint reinforce the supportive mechanism. The side pockets on each side of the brace holds the hinge in place. The hinges of the brace play a critical role, each hinge moves freely through full range of flexion and extension and can be locked manually in full extension. The locking mechanism of the brace maintains the knee joint in full extension and gives the patient the ability to stand and walk. The two flexible stays assist the knee joint and prevent the excessive extension. The SUPPORTIVE KNEE BRACE is light weight and easy to use.

### BACKGROUND OF THE INVENTION

This invention relates to a supportive knee brace which is invented primarily to establish the locking mechanism in the knee joint when the extensory apparatus is limited or weakened by paralysis or equilibrium deficiency. Many orthopaedic braces exist for different medical uses. Some are used to support the knee during sport activity, some are used after knee surgery to protect the ligament reconstruction during the recovery period and some are used to reduce the muscle contracture in the knee joint. None of these braces provides a means to support the knee during standing and walking when the knee extensors are weak or paralyzed. The Supportive Knee Brace is a simplified brace that can provide the sufficient support to the knee joint and enable the patient to walk without the fear of falling.

### SUMMARY OF THE INVENTION

I have invented the Supportive Knee Brace to be used as a supportive device for patient with severe muscle weakness or paralysis in the knee joint. My brace is made with soft material called neoprene and has a soft contact with the patients skin. The Supportive Knee Brace has a longitudinal pocket on each side for insertion of the hinged rod. The mid section of the hinge is placed on the medial and lateral axis of the knee joint and moves through the fall range of flexion and extension. The hinge has a lock button and can be locked and unlocked manually by the patient. The Supportive Knee Brace has two straps above and below the knee which can reinforce the locking position during walking. Two flexible stays of the brace located on the back of the brace support the knee joint against excessive extension movement in the joint.

### A BRIEF DESCRIPTION OF THE DRAWINGS

FIG. 1 is a top plan view of the Supportive Knee Brace.

FIG. 2 is a bottom plan view for the Supportive Knee Brace.

FIG. 3 is a perspective view of the Supportive Knee Brace positioned on a patient's knee in full locking position.

FIG. 4 is a view of a hinged support element in full extension and in a 90 degree bending position.

FIG. 5 shows two flexible stays.

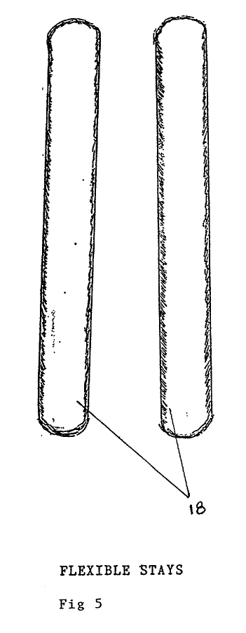

### DETAILED DESCRIPTION OF THE INVENTION

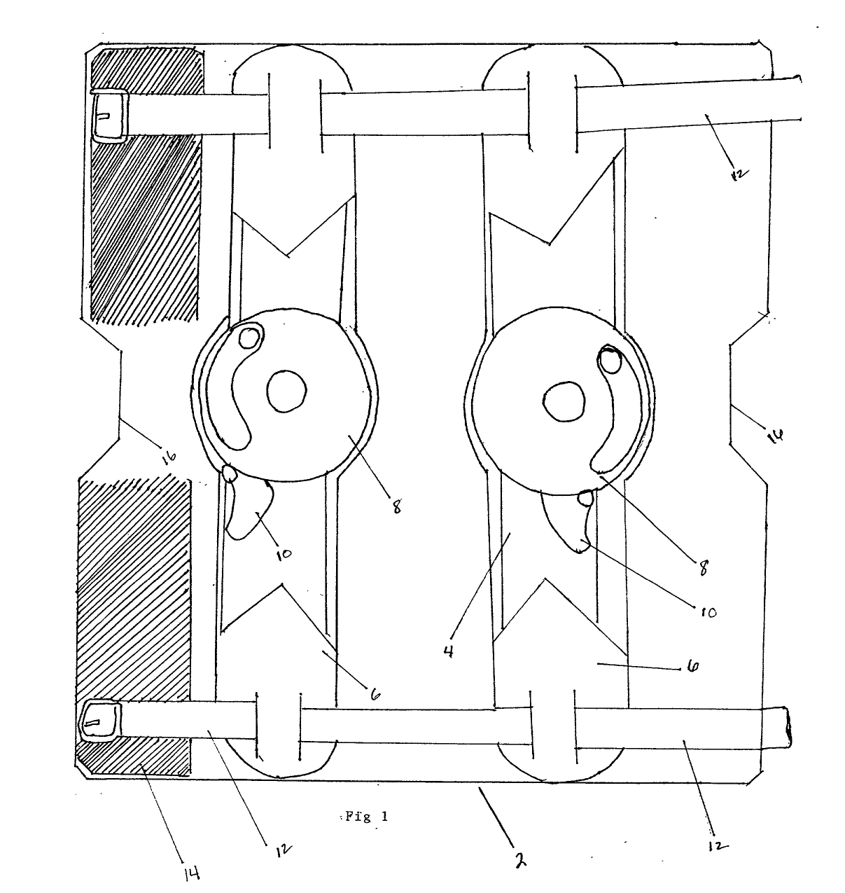

FIG. 1. The Supportive Knee Brace 2 is shown presenting the top plan view. The brace is made with soft material called neoprene. The edge of the Brace 2 has a velcro section 14 which is sewn securely to the edge of the knee brace, above and below the patella opening 16. Strap 12 passes through pocket 6 on top and bottom of the knee brace 2 are used in fastening the knee brace during use.

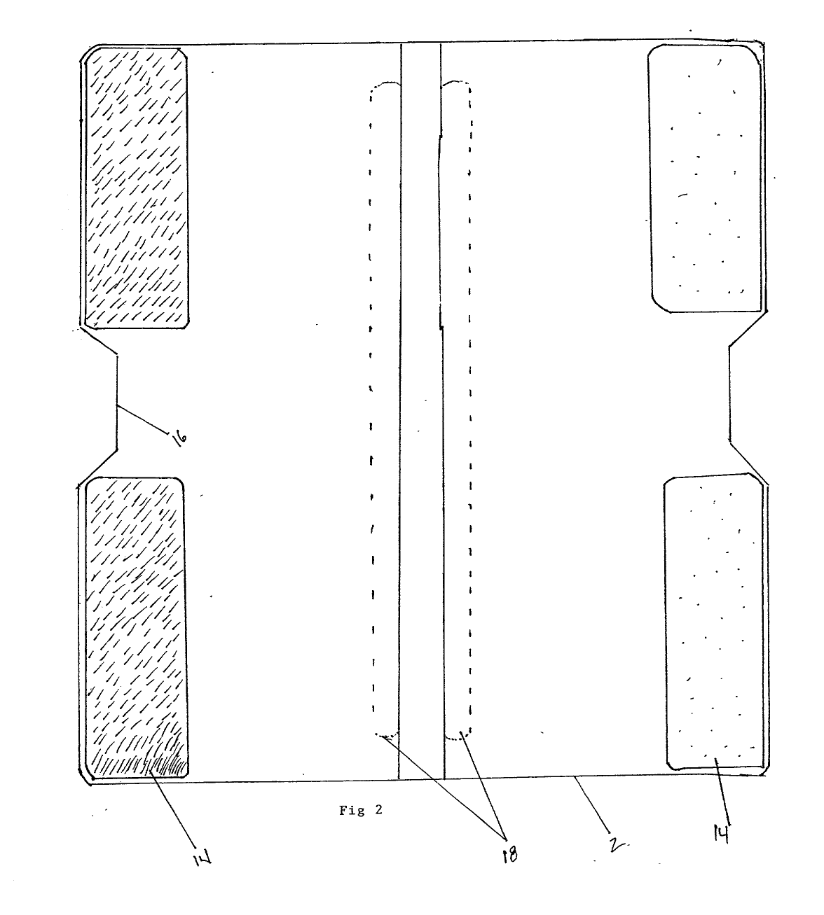

FIG. 2. The Supportive Knee Brace 2 is shown presenting a bottom plan view of the Supportive Knee Brace. On the mid section of the brace 2 are flexible stays 18 which are inserted in the longitudinal position and fit s the contour section of the knee joint on the back and give an additional support during standing and walking.

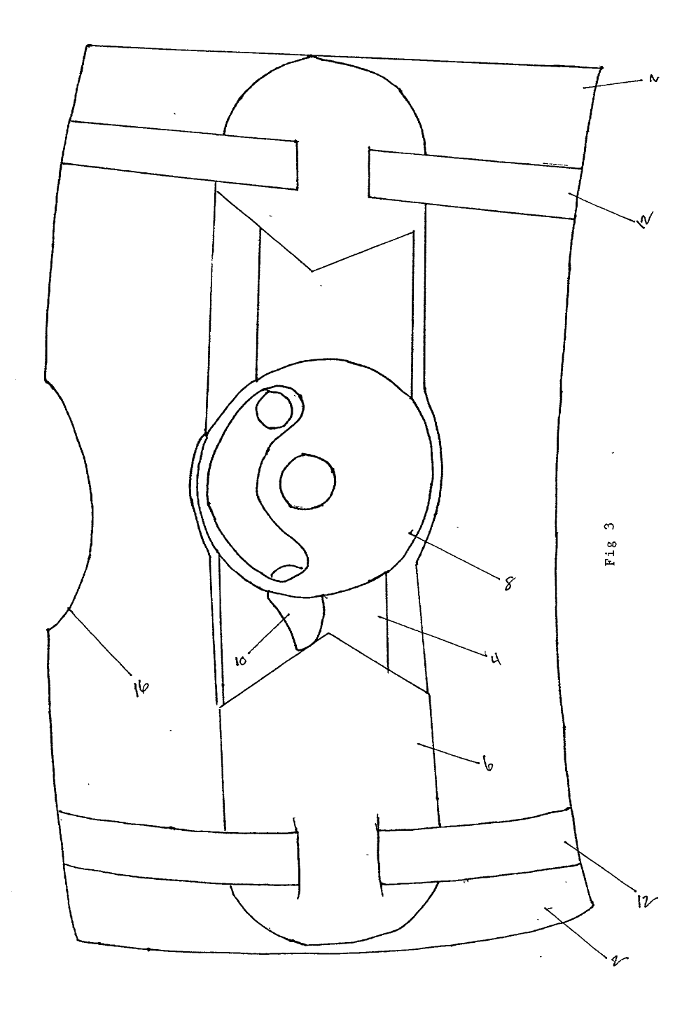

FIG. 3. The Supportive Knee Brace 2 is shown presenting a view of the knee brace positioned on a patients knee with the hinged rod in a straight and locking position.

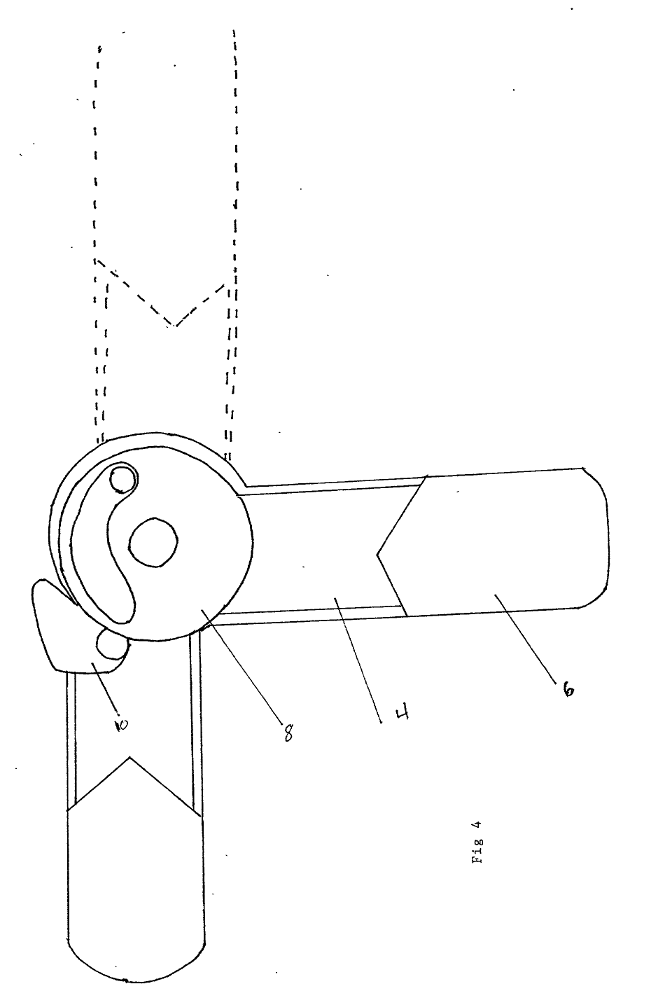

FIG. 4. The Supportive Knee Brace 2 shown presenting a view of a hinged rod 6 in 90degrees and 180degrees position. The hinged rod is placed in pocket 4 which is securely holding in stable position. When the Supportive Knee Brace is position on the patient's knee, the circular section 8 of the rod 6 are placed precisely over the medial and lateral section of the knee joint. The hinged rods are capable of maneuvering to the full range of flexion and extension position and it can be manually locked by locking element 10 in a straight position for standing or walking.

1. An orthopedic appliance for surrounding a knee of a patient to assist weak extensory muscle capability, the appliance comprising:
A body having a cloth outer layer and a foam inner layer for contact with the skin of the patient, the body being adapted to be wrapped around a knee of a patient such that an area is provided from which a patella portion of the knee can protrude, means attached to the body for securing the body above and below the patella portion of the knee in a wrapped position about the knee, two hinged support elements, each support element having ends received in a pair of the longitudinal end pockets on one of the lateral sides of the body, and a pair of stays attached to the body interior of the pairs of oppositely positioned end pockets so as to be positioned on the posterior side of the knee to assist the extensory muscles to resist excessive extension during standing and walking.

2. The orthopedic appliance according to claim 1, wherein the support element received in each pair of end pockets is hinged and includes a full extension stop to permit the patient's knee to be limited in full extension position which helps the patient to stand and walk without the collapsing of the knee joints.
